(19) United States
(12) Patent Application Publication
(10) Pub. No.: US 2008/0262580 A1
(43) Pub. Date: Oct. 23, 2008

(54) HEATING IMPLANTABLE DEVICE TO TREAT A CONDITION

(75) Inventors: Martin T. Gerber, Maple Grove, MN (US); John C. Rondoni, Plymouth, MN (US)

Correspondence Address: CAMPBELL NELSON WHIPPS, LLC, 408 ST. PETER STREET, SUITE 240, ST. PAUL, MN, 55102, US

(73) Assignee: MEDTRONIC, INC., Minneapolis, MN (US)

(21) Appl. No.: 12/023,074

(22) Filed: Jan. 31, 2008

Related U.S. Application Data
(60) Provisional application No. 60/912,708, filed on Apr. 19, 2007.

(51) Int. Cl. A61F 7/12

(52) U.S. Cl. 607/113

(57) ABSTRACT

An implantable medical device includes a housing and electronics disposed in the housing. The device also includes a heating element disposed in proximity to the housing and operably coupled to the electronics. The electronics are configured to control heating of the heating element to heat tissue in proximity to the housing to aid in the treatment of a condition of the patient in proximity to the device when implanted. The device may further include a temperature sensor disposed in proximity to the housing and operably coupled to the electronics. In such embodiments, the electronics may be configured to control the amount of heat generated by the heating element based on information transmitted from the temperature sensor.

This application claims the benefit of Provisional Application Ser. No. 60/912,708, filed on Apr. 19, 2007, which application is hereby incorporated by reference in its entirety to the extent that it does not conflict with the disclosure presented herein.

### FIELD

This disclosure relates, inter alia, to implantable rechargeable medical devices. More particularly, it relates to systems, devices and methods for heating implantable medical devices to aid in treatment of a condition, such as infection, in proximity to medical devices implanted in patients.

### BACKGROUND

The medical device industry produces a wide variety of electrical and mechanical devices for treating patient medical conditions. For some medical conditions, medical devices provide the best, and sometimes the only, therapy to restore an individual to a more healthful condition and a fuller life. Examples of implantable medical devices include neurostimulators, infusion devices, pacemakers, defibrillators, diagnostic recorders, and cochlear implants. While such devices may vary in their mechanisms of therapeutic or diagnostic action or their therapeutic or diagnostic target, there are several common health concerns associated with the use of such devices.

For example, infection may result following implantation. Today, infections associated with implanted medical devices are not very common due to care and precautions taken during surgical implantation of the devices. However, when infection associated with an implanted medical device does occur, explanting the device is often the only appropriate course of action.

Other heath issues that may arise following implantation include pain, seromas, hematomas, and edema in proximity to the implanted device. All of which can present significant risk or discomfort to the patient into which the device is implanted.

### SUMMARY

The present disclosure describes, inter alia, systems, devices and methods that can be used to aid in treatment of a condition, such as infection, in proximity to medical devices implanted in patients. The methods, systems and devices heat tissue in proximity of the device to facilitate treatment.

In various embodiments, an implantable medical device is described. The device includes a housing and electronics disposed in the housing. The device also includes a heating element disposed in proximity to the housing and operably coupled to the electronics. The electronics are configured to control heating of the heating element to heat tissue in proximity to the housing to aid in the treatment of a condition of the patient in proximity to the device when implanted. The device may further include a temperature sensor disposed in proximity to the housing and operably coupled to the electronics. In such embodiments, the electronics may be configured to control the amount of heat generated by the heating element based on information transmitted from the temperature sensor.

In various embodiments, a method for treating a condition of a patient in proximity to an implanted medical device is described. The method includes detecting the condition in the patient in proximity to the implanted device and heating the device to facilitate treatment of the condition. Heating the device may include activating a heating element disposed in proximity to a housing of the device.

By providing devices, systems and methods that heat tissue in proximity to an implanted medical device, health conditions of the patient in proximity to or associated with the device may be better treated. Conditions that may benefit from heat treatment include infection, pain or discomfort, edema, seromas and hematomas. Use of the implanted device to provide heat treatment may facilitate clearing the condition and prevent potentially dangerous device explant. These and other advantages will be readily understood from the following detailed descriptions when read in conjunction with the accompanying drawings.

### BRIEF DESCRIPTION OF THE DRAWINGS

FIG. 1 is a diagrammatic representation of a perspective view of an environment of a rechargeable system including a rechargeable medical device implanted in a patient.

FIG. 2 is a schematic block diagram of representative components of a illustrative rechargeable implantable medical device.

FIG. 3 is a schematic block diagram of representative components of a illustrative rechargeable implantable medical device.

FIG. 4 is a schematic of a perspective view of an illustrative implantable medical device.

FIG. 5 is a schematic of an exploded perspective view of a housing of an illustrative implantable medical device.

FIG. 6 is a top view of a resistive wire heating element disposed on a surface of an implantable medical device.

FIGS. 7 and 8 are flow diagrams of illustrative methods.

The drawings are not necessarily to scale. Like numbers used in the figures refer to like components, steps and the like. However, it will be understood that the use of a number to refer to a component in a given figure is not intended to limit the component in another figure labeled with the same number.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope of spirit of the present invention. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

“Condition”, “disease”, “disorder”, or the like are used herein interchangeably.

The present disclosure describes, inter alia, systems, devices and methods that can be used to heat tissue in proximity to an implanted device to facilitate in treatment of a condition, such as infection. While not intending to be bound by theory, it believed that heat generated in proximity to the implanted device will cause increased circulation and associated healing of various conditions that may be associated with the implanted device. Increased circulation may result in increased delivery of therapeutic agents, if taken, to aid in treatment of the condition. In some embodiments, heat may be used to ablate tissue, such as infected tissue, to facilitate treatment of the condition.

In various embodiments, methods, systems and devices are described in which it is determined whether an infection is in proximity to an implanted medical device. If an infection is determined to be present, the device may be heated to facilitate clearing of the infection.

In various embodiments, methods, systems and devices are described in which temperature in proximity of an implantable rechargeable device is monitored in connection with heating the device. If the monitored temperature falls outside a desired range, one or more parameters associated with heating are modified to cause the temperature to reside within the desired range. The desired temperature range, in various embodiments, is a range that can facilitate treatment of a condition in proximity to the implanted device without causing undesired damage to the patient's tissue surrounding the implanted device.

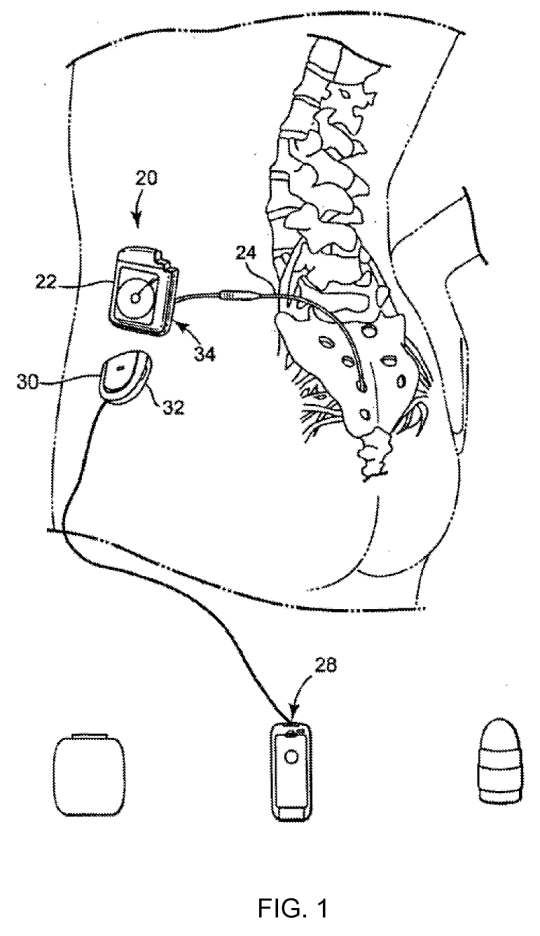

Referring to FIG. 1, the general environment of an embodiment of a rechargeable implantable medical device 20 is shown. An implantable electrical signal generator 22 is shown in FIG. 1, but other embodiments such as drug delivery pumps, pacemakers, defibrillators, diagnostic recorders, cochlear implants, and the like are also applicable. Implantable medical devices 20 are often implanted subcutaneously approximately one centimeter below the surface of the skin with an associated therapy delivery element, such as an electrical lead 24 or catheter, extending to one or more therapy sites. Rechargeable implantable medical device 20 is recharged with a recharging device 28 such as a patient charger or programmer that also has a charging capability.

Recharging an implantable medical device 20 generally begins with placing a recharging head 30 containing a primary recharging coil 32 against the patient's skin near the proximal side of the medical device 20. Some rechargers 28 have an antenna locator that indicates when recharge head 30 is aligned closely enough with implanted medical device 20 for adequate inductive charge coupling. The recharge power transfer signal is typically a frequency that will penetrate transcutaneous to the location of implanted medical device 20 such as a frequency in the range from 5.0 KHz to 100 KHz. The power transfer signal is converted by implantable medical device 20 into regulated DC power that is used to charge a rechargeable power source 34. Telemetry can also be conducted between the recharger 28 and the implanted medical device 20 during recharging. Telemetry can be used to aid in aligning recharger 28 with the implanted medical device 20, and telemetry can be used to manage the recharging process. Telemetry is typically conducted at a frequency in the range from 150 KHz to 200 KHz using a medical device telemetry protocol, but may also include Bluetooth®, 802.11, and Medical Implant Communication Service (MICS) frequency band communication. For telemetry, the recharger 28 and implanted medical device 20 typically have a separate telemetry coil. Although, the recharging coil can be multiplexed to also serve as a telemetry coil.

While device 20 shown in FIG. 1 is rechargeable, it will be understood that the teachings presented herein apply to devices 20 that are not rechargeable. However, due to power consumption associated with heating of device 20, described in more detail below, it may be desirable for device 20 to include a rechargeable power source.

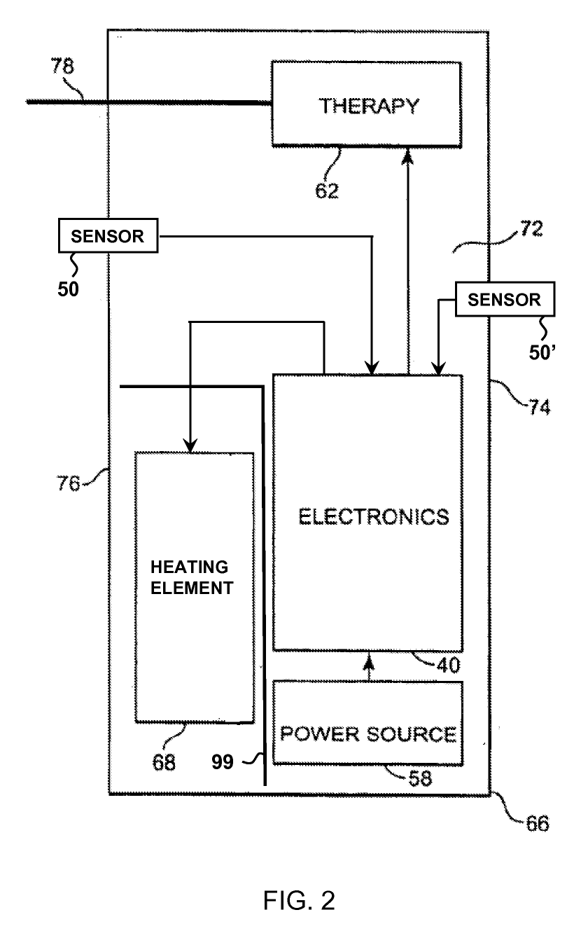

Referring to FIG. 2, a schematic diagram of an implantable medical device 20 having a heating element 68 is shown in block form. Implantable medical device 20 also includes a housing 66, electronics 40, a power source 58, and one or more sensors 50, 50′. Housing 66 has an interior cavity 72, an exterior surface 74, a proximal face 76, and a therapy connection 78. The therapy connection 78 can be any type of therapy connection 78 such as a stimulation feedthrough, a drug infusion port, or a physiological sensor. There can also be more than one therapy connection 78 and a combination of different types of therapy connections 78. Housing 66 is hermetically sealed and manufactured from a biocompatible material such as titanium, epoxy, ceramic, and the like.

Electronics 40 are carried in the housing interior cavity 72 and configured to perform a medical therapy or diagnostic. Electronics 40 are operably coupled therapy module 62, heating element 68, and sensors 50, 50′. Power source 58 is carried in the housing interior cavity 72 and coupled to electronics 40. Power source 58 can be a physical power source such as a spring, an electrical power source such as a capacitor, or a chemical power source such as a battery. The battery can be a hermetically sealed rechargeable battery such as a lithium ion (Li+) battery or the like.

Heating element 68 is coupled to electronics 40 and can also be coupled to power source 58 in addition to electronics 40. In various embodiments, the heating element 68 can be located on housing proximal face 76, inside housing 66 (e.g., as shown in FIG. 2), and remotely away from housing 66. Heating element 68 may be any suitable heating element. For example, the heating element 68 may include power electronics, such as power transistors, designed in a configuration to dissipate minimal heat or resistive wires. In various embodiments, heating element 68 is a resistive wire and may be manufactured from, for example, nickel chromium or other suitable material. Heating element may be located in contact with housing 66 to allow for heating of tissue on the exterior surface of the device 20. In various embodiments, heating element is a resistive wire that is weaved, coiled, or the like around the inside of the housing 66 to provide substantially even heating across the surface of the housing 66 when heated by heating element 68. In various embodiments (not shown), the heating element 68 may be the housing 66. For example, if the housing a titanium housing, signals that interact with metallic impurities in the housing 66 may be employed to heat the housing 66. Electronics 40 or other device components may be thermally insulated from heating element 68; e.g., by a heat insulating shield 99. Insulating shield 99 may be formed from any suitable material, such as a ceramic material, foam, or fiberglass fiber.

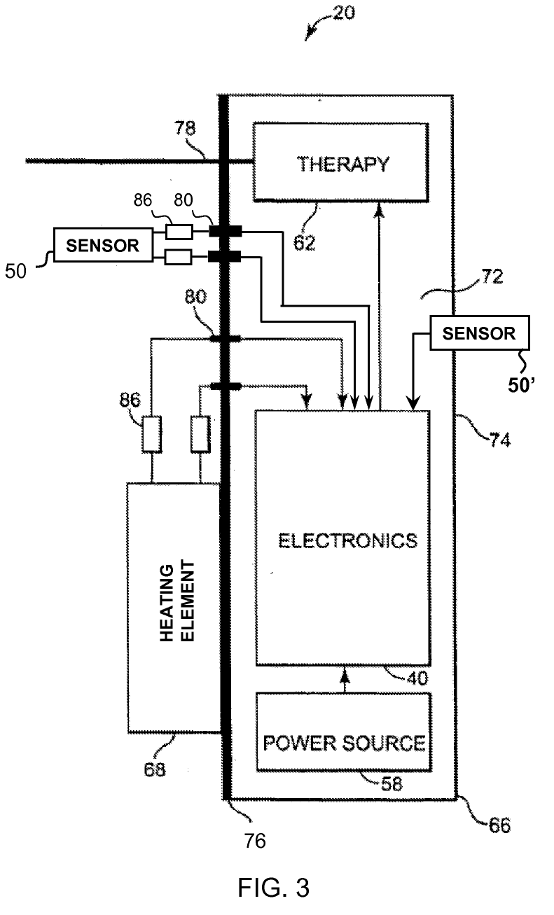

Referring to FIG. 3, heating element 68 may be disposed external to housing 66; e.g. disposed on housing proximal face. Feedthroughs 80 may be employed to operably couple heating element 68 to electronics 40, while maintaining the hermetic seal of housing 66. Heating element 68 is connected to feedthroughs 80 with an electrical connection 86 that is hermetically sealed to prevent electrical connection 86 from being exposed to body tissue or fluid when implanted. Proximal face 76 of device 20 may include thermal insulating material to prevent internal components of device 20 from potentially adverse effects of heat generated by heating element 68.

Referring now to both FIGS. 2 and 3, one or more sensors 50, 50′ may be coupled to electronics and are disposed in or on, generally in proximity to, device 20 or portion thereof. Sensor 50, 50′ may be exposed to an external surface of device 20 to be in contact with body tissue or fluid when implanted in a patient, or may be contained in housing 66, as appropriate. Sensors 50, 50′ may be used to monitor temperature of device 20 or tissue in proximity to device 20, or to monitor a patient condition associated with or in proximity to the device 20.

In general, sensor 50, 50′ may be any device capable of detecting and transmitting information to device 20. If housing 66 is hermetically sealed, feedthroughs 80 may be used to provide electrical connectivity through housing 66 while maintaining the hermetic seal. While not shown, it will be understood that one or more sensor capable of detecting an indicator of infection or other condition of a patient may be located on, in, or about accessory therapeutic element, such as a catheter or lead 24. In various embodiments, sensor 50, 50′ is capable of detecting information regarding an indicator of infection or other condition or is capable of detecting and transmitting information that may be useful in determining whether an indicator of infection or other condition may actually be indicative of infection. Additional information regarding such sensing and use of such information in systems including implantable medical devices is provided in (i) U.S. patent application Ser. No. 11/737,180, entitled “INDICATOR METRICS FOR INFECTION MONITORING”, filed on Apr. 19, 2007; and (ii) U.S. patent application Ser. No. 11/737,181, entitled “Multi-Parameter Infection Monitoring”, filed on Apr. 19, 2007, which applications are hereby incorporated herein by reference in their respective entireties to the extent they do not conflict with the disclosure presented herein. Examples of physical or chemical stimuli that may serve as indicators of infection are temperature, impedance, pH, and biological markers of infection. Examples of parameters that may be provide information useful for determining whether an indicator of infection may actually be indicative of infection include parameters indicative of patient activity.

In addition to being monitored as an indicator of infection, temperature may be monitored in connection with heating of device 20 via heating element 68 to determine whether temperature is in a desired range. If temperature is not within the desired range, one or more parameters associated with the heating, such as amplitude of current through heating element 68, may be modified to encourage temperature in proximity to the surface of device 20 to return to the desired range. Any suitable sensor 50, 50′ capable of detecting temperature or changes in temperature may be employed. For example, temperature sensor 50, 50′ may include a thermocouple, a thermistor, a junction-based thermal sensor, a thermopile, a fiber optic detector, an acoustic temperature sensor, a quartz or other resonant temperature sensor, a thermo-mechanical temperature sensor, a thin film resistive element, or the like.

The use of more than one temperature sensor at different locations may serve to improve the accuracy of determinations as to whether temperature at a given sensor location is indicative of infection or other condition by comparing the temperature at the given location to temperature at a location removed from the given location. Additional information regarding the use of temperature sensors at two locations for improved infection monitoring is described in U.S. patent application Ser. No. 11/737,171, entitled “Implantable Therapy Delivery System Having Two Temperature Sensors For Infection Monitoring”, filed on Apr. 19, 2007, which application is incorporated herein by reference in its entirety to the extent that it does not conflict with the disclosure presented herein. Of course sensor 50′ may detect indicators of infection or physical or chemical stimuli other than temperature.

While two sensors 50, 50′ are shown in FIGS. 2 and 3, it will be understood that any number of temperature or other sensors may be employed, such as those described in (i) U.S. patent application Ser. No. 11/737,180, entitled “INDICATOR METRICS FOR INFECTION MONITORING”, filed on Apr. 19, 2007; and (ii) U.S. patent application Ser. No. 11/737,181, entitled “Multi-Parameter Infection Monitoring”, filed on Apr. 19, 2007.

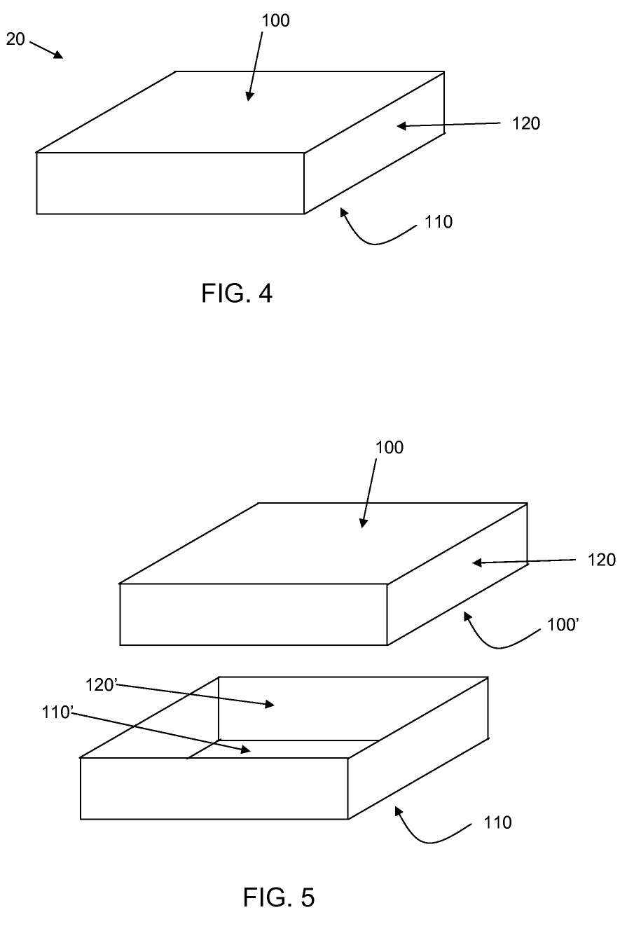

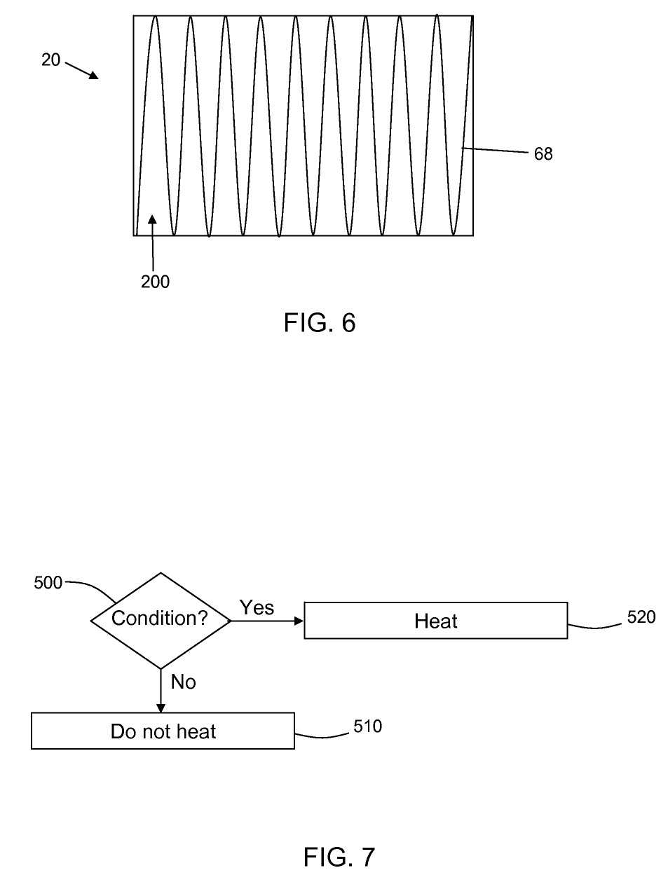

In the illustrative embodiment shown in FIGS. 4 and 5, device 20 has first 100 and second 110 opposing major exterior surfaces and edge exterior surface 120. Device 20 also has first 100′ and second 110′ opposing interior surfaces and interior edge surface 120. Heating element (not shown in FIGS. 4 and 5) may be disposed at any location on, in, or about (generally in proximity to) any one or more surfaces of device. For example, and referring to FIG. 6, a resistive wire heating element 68 may be disposed on one or more surface 200 of device 20 in a manner that allows for relatively uniform heating along the surface 200 when current is applied to the resistive wire via the device electronics.

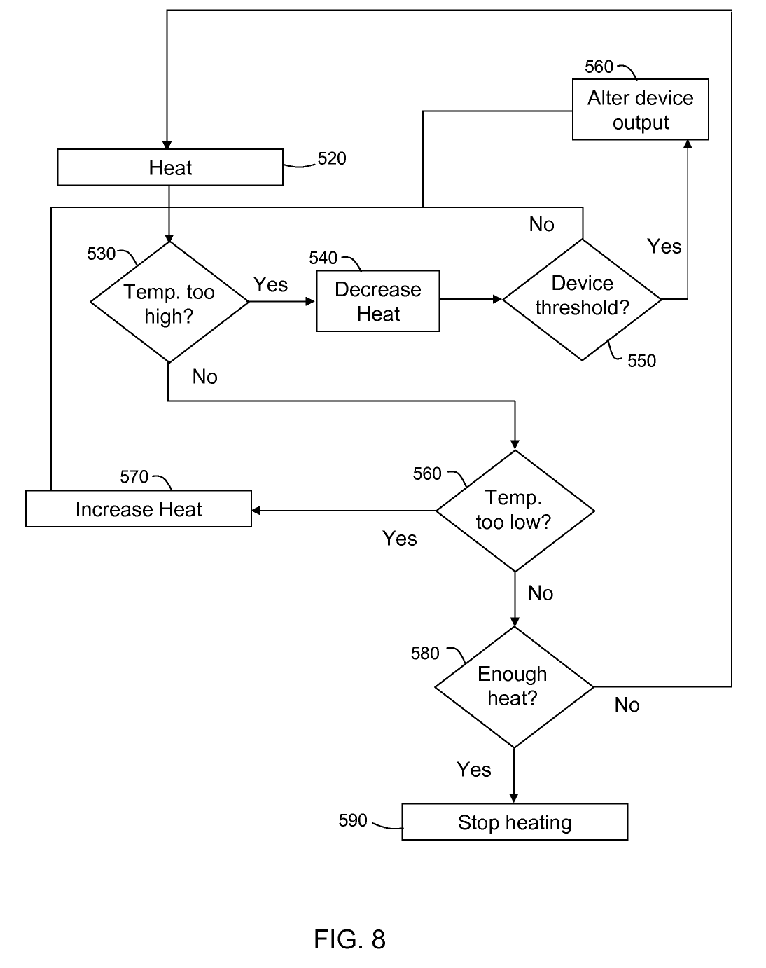

It will be understood that the components described in FIGS. 1-6 are but examples of components that an implantable device 20 may include and that many other device or system configurations may be employed to carry out the methods described below. However, for the sake of convenience, the discussion that follows with regard to the methods illustrated in the flow diagrams of FIGS. 7 and 8 will refer to components as described with regard to FIGS. 1-6.

Referring to FIG. 7, a flow diagram of a representative method is shown. According to various embodiments, the method includes determining whether a condition associated with the device 20 is present in the patient in proximity to the device 20 (500). Any condition for which heat treatment may be beneficial may be detected. Such conditions include pain, infection, hematoma, seroma, and edema. If it is determined that no condition is present, heat is not applied via heating element 68 (510). If a condition is detected, heat is applied via heating element 68 (520). The determination (500) may be made in whole, or in part, by device 20 using, for example, information obtained by sensors 50, 50′. Alternatively, the determination (500) may be made by a clinician or other health care provider via examination of the patient. A telemetric or other suitable signal may be sent to the implanted device 20 instructing electronics 40 to activate heating element 68.

Referring to FIG. 8, a flow diagram of a representative method for controlling heat production via heating element 68 is shown. Heat is applied to tissue or device 20 via heating element 68 (520), and a determination is made as to whether the temperature is too high (530) through, for example, the use of temperature sensor 50, 50′. If temperature is too high, heating is decreased (540) by, for example, reducing current applied to a resistive wire heating element 68. The method may include determining whether a device threshold has been crossed (640) and altering device output (650) if the threshold is crossed. Such steps may be desirable when device 20 is an infusion device or other device, where increased temperatures may result in delivery of excess or inappropriate levels of therapy. As such, device output may be altered based on monitored temperature in proximity to the device. For example, the rate of delivery of therapeutic agent may be appropriately lowered or altered, depending on the monitored temperature. In various embodiments, therapeutic output may be temporarily suspended until temperature in proximity to the device returns to an acceptable range.

An additional concern with implantable drug infusion devices is that increased temperatures may result in degradation of therapeutic agent to be delivered by the infusion device. One skilled in the art will be able to balance the desire for therapeutic heat for treatment of a condition associated with an implanted device and the potential adverse effects on the therapy and therapeutic agent to make a decision regarding what the device threshold may be on a case-by-case, therapy-by-therapy, or therapeutic agent-by-therapeutic agent basis.

If temperature is not too high, a determination may be made as to whether temperature at device 20 or tissue is too low (560) for purposes of treating the condition. If temperature is too low, heating is increased (570) by, for example, increasing current applied to a resistive wire heating element 68. If temperature is not too low, a determination may be made as to whether sufficient heat has been applied to obtain therapeutic benefit from the heat (580). The determination as to sufficient heat (580) may be made prior to beginning the heating process based on, for example, a predicted amount of heat that may be beneficial for treatment of the detected condition. Alternatively, or in addition, the determination as to sufficient heat (580) may be made during treatment based on, for example, information obtained from sensors 50, 50′ regarding the state of the condition. If enough heat has not been applied, heating (520) is continued. If enough heat has been applied, heating is stopped (590).

Generally, it is considered desirable to minimize heat production from an implantable medical device 20. However, detection of a condition such as infection in proximity of the device places the patient safety at risk and more aggressive heating may be warranted. For example, it may be desirable for temperature at a surface 200 of the implanted device 20 to rise 2° C., 5° C., 7° C., 10° C. or more from a temperature prior to heating, perhaps for the majority of the time that the heating element 68 is activated. In some embodiments, it may be desirable to pulse heating. For example, it may be desirable to cause temperature at a surface 200 of the implanted device 20 to rise more than 2° C., 5 C, 7° C., or 10° C. from a temperature prior to heating, return to within 2° C., 5° C., 7° C., or 10° C. of the temperature prior to heating, and then rise more than 2° C., 5° C., 7° C., or 10° C. from a temperature heating. In some embodiments, it may be desirable to cause tissue ablation in proximity of the device 20. The extent of tissue ablation may be controlled by the rate of the heating of the device 20. For example, a rapid quick rise in amplitude of current applied to a resistive wire heating element 68, followed by a rapid quick decrease, may allow for ablation of a small amount of tissue. On the other hand, sustained elevated amplitude may result in greater tissue ablation. Generally, tissue ablation will occur with a temperature increase of 5° C. to 10° C., depending on the amount of time heating element 68 is held at the increased temperature. Even higher temperature increases may be needed if the duration of the temperature increase is short. Ablation of tissue may be desirable in instances where tissue in proximity to the implanted rechargeable device 20 is infected.

One of skill in the art will understand that components or steps described herein regarding a given embodiment or set of embodiments may readily be omitted, substituted, or added from, with, or to components or steps of other embodiments or sets of embodiments, as appropriate or desirable.

It will be further understood that a computer readable medium containing instructions that when implemented cause an implantable medical device (or system including an implantable medical device) to perform the methods described herein are contemplated. Devices and systems including the computer readable medium are also contemplated.

Patent applications that include discussion to infection monitoring that may provide additional insight into the teachings provided herein include the following patent applications filed on Apr. 19, 2007: (i) U.S. patent application Ser. No. 11/737,173, entitled “Infection Monitoring”; (ii) U.S. patent application Ser. No. 11/737,170, entitled “Infection Monitoring”; (iii) U.S. patent application Ser. No. 11/737,169, entitled “Event Triggered Infection Monitoring”; (iv) U.S. patent application Ser. No. 11/737,179, entitled “Controlling temperature during recharge for treatment of a condition”; and (v) U.S. patent application Ser. No. 11/737,176, entitled “Refined Infection Monitoring”. The above-referenced patent applications are hereby incorporated herein by reference in their respective entireties to the extent that they do not conflict with the disclosure presented herein.

Thus, embodiments of HEATING IMPLANTABLE DEVICE TO TREAT A CONDITION are disclosed. One skilled in the art will appreciate that the present invention can be practiced with embodiments other than those disclosed. The disclosed embodiments are presented for purposes of illustration and not limitation, and the present invention is limited only by the claims that follow.

What is claimed is:

1. An implantable medical device comprising:
a housing;
electronics disposed in the housing; and
a heating element disposed in proximity to the housing and operably coupled to the electronics,
wherein the electronics are configured to control heating of the heating element to heat tissue in proximity to the housing to aid in the treatment of a condition of the patient in proximity to the device when implanted.

2. The device of claim 1, further comprising a temperature sensor disposed in proximity to the housing and operably coupled to the electronics, wherein the electronics are configured to control the amount of heat generated by the heating element based on information transmitted from the temperature sensor.

3. The device of claim 2, wherein the electronics are configured to determine whether a condition is present in the patient based on information received from the temperature sensor.

4. The device of claim 3, wherein the electronics are configured to activate the heating element if a determination is made that the condition is present.

5. The device of claim 1, wherein the heating element comprises a resistive wire.

6. The device of claim 1, wherein the heating element is disposed external to the housing.

7. The device of claim 6, wherein the heating element comprises a resistive wire.

8. The device of claim 7, wherein the resistive wire is disposed about the housing.

9. The device of claim 1, wherein the heating element is disposed internal to the housing.

10. The device of claim 9, further comprising a heat insulating shield disposed between the electronics and the heating element.

11. The device of claim 1, wherein the housing is the heating element.

12. A method for treating a condition of a patient in proximity to an implanted medical device, the method comprising:
detecting the condition in the patient in proximity to the implanted device; and
heating the implantable device or a component thereof to cause heating of tissue in proximity to the implanted device to facilitate treatment of the condition.

13. The method of claim 12, wherein heating the device comprises activating a heating element disposed in proximity to a housing of the device.

14. The method of claim 12, wherein detecting the condition comprises sensing an indicator of the condition in proximity to the device and providing information regarding the sensed indicator to electronics of the device.

15. The method of claim 12, further comprising sensing temperature in proximity to the device and modifying the heating based on the sensed temperature.

16. The method of claim 12, wherein detecting the condition comprises sensing temperature in proximity to the device.

17. A computer readable medium containing instructions that when implemented cause an implantable medical device to:
detect a condition in a patient in proximity to the device when implanted; and
heat tissue in proximity to the implanted device to facilitate treatment of the condition.

18. An implantable medical device comprising:
a heating element capable of heating tissue in proximity to the device when implanted;
electronics operably coupled to the heating element; and
a sensor operably coupled to the electronics and capable of detecting condition of a patient in proximity to the device when implanted; and
a computer readable medium according to claim 17 readable and executable by the electronics.
